(19) United States
(12) Patent Application Publication
(10) Pub. No.: US 2011/0098654 A1
(43) Pub. Date: Apr. 28, 2011

(54) Anchoring And Protective Intravenous Shield

(76) Inventor: Russell Shipman, York, ME (US)

(21) Appl. No.: 12/706,445

(22) Filed: Feb. 16, 2010

Related U.S. Application Data
(60) Provisional application No. 61/255,270, filed on Oct. 27, 2009.

(51) Int. Cl. A61M 25/02

(52) U.S. Cl. 604/180

(57) ABSTRACT

An anchoring protection device for covering and protecting an intravenous access site comprises an intravenous compartment hub portion, coupled to a U-shaped portion coupled to a straight tubing portion. The combination of the compartment hub portion, U-shaped portion and straight tubing portion allows for an area where the intravenous catheter, catheter needle, luer lock and tubing can be placed, surrounding which is a flat, generally planar area that includes an adhesive backing for adhering the device to the skin of a patient. The anchoring protection device is a one-piece unit that is made from a thin, transparent, flexible material.

### CROSS REFERENCE TO RELATED APPLICATIONS

This application is related to and claims priority under 35 USC §119(e) to U.S. provisional Application No. 61/255,270 filed Oct. 27, 2009 entitled “Anchoring and Protective Intravenous Shield”, which is incorporated fully herein by reference.

### TECHNICAL FIELD

The present invention relates to a protective anchoring device and more particularly, relates to a shield for protecting and securing an intravenous catheter.

### BACKGROUND INFORMATION

The need to protect the intravenous site during medical treatment is well known. An intravenous catheter needs to be protected from several adverse events. These events include intravenous catheter dislodgement causing infiltration of intravenous fluids as well as contamination from airborne sources that may lead to infections or disruption of a needed moisture barrier. The intravenous site is at risk for unintended or purposeful dislodgement, which will interrupt the fluid administration. This interruption will lead to delay of medical treatment, possible pain and infection in the patient, increased nursing time and higher medical costs due to the need to reinsert the intravenous line and due to the possibility of prolonged treatment time in the hospital.

The prior art focuses on securing and protecting the intravenous area in the adult population. The prior art does not deal with the pediatric and geriatric population. The pediatric and geriatric population is at higher risk of the patient grabbing and pulling at the intravenous tubing resulting in dislodgment of the intravenous catheter. Therefore, the prior art devices require improvements in order to solve the problems caused by use with all patients and in particular, in pediatric and geriatric patients.

A number of devices in the prior art disclose securing, protecting and allowing for visualization of the intravenous site. For example, in U.S. Pat. No. 4,669,458 to Abraham et al. and U.S. Pat. No. 6,809,230 to Hancock, a clear plastic window is adhesively applied to the IV site. U.S. Pat. No. 5,112,313 to Sallee and U.S. Pat. No. 5,116,324 to Brierley disclose a plastic cover over the IV site. These patents disclose nonflexible devices that allow the covering to be raised to allow inspection of the insertion site. Other patents such as U.S. Pat. No. 6,322,539 to Cook are intravenous guards shaped like animals to protect the IV site. They are held in place by a wristband. Other prior art patents are U.S. Pat. No. 503,973 to Lovejoy, U.S. Pat. No. 5,342,317 to Claywell and U.S. Pat. No. 6,228,064 to Abita. These patents disclose methods and apparatus for holding the intravenous catheter in place using a strap or band that may or may not be adhered to skin.

There are a number of disadvantages to each of the inventions in the prior art. The ability for the intravenous catheter to slide out of the venous or arterial lumen, thereby causing dislodgement and infiltration of fluids within the subcutaneous space leads to a caustic painful reaction and possible infection. Prior art issues also include lack of safety in the pediatric population, membranes that can be interrupted by tearing, and covers that do not anchor the I.V. catheter in place in a one-piece method. Therefore, there exists a need for a one-piece system and method of anchoring and protecting an intravenous catheter while also allowing for visual assessment of the intravenous catheter.

### SUMMARY

This invention relates to a shield for protection of a peripheral venepuncture site for patients requiring intravenous fluids. In an exemplary embodiment of the present invention, the protection device allows for an area to be molded around an intravenous compartment hub, a lure lock and tubing as it sits on top of the skin. The molded area around the intravenous compartment hub allows for a flat surrounding area that facilitates anchoring and stabilization and specifically for preventing movement of the intravenous catheter out of the venous or arterial lumen.

In the exemplary embodiment of the present invention, the shield protects the intravenous site from being manipulated or tampered with. In one embodiment, a urethane material will be used for the shield, which is rip proof and access proof. The material will be resistant to tearing of the cover, thereby causing dislodgement of the intravenous catheter. The combination of the unique material and the anchoring quality prevents removal.

In another embodiment of the present invention, the backing of the shield is coated with an adhesive backing that is scored to make at least two removable pieces. In a further embodiment of the present invention, the adhesive backing is scored into two equal halves, with the score line running down a centerline of the backing of the shield. The adhesive backing surrounds the body of the intravenous compartment hub and is fixably attached to the skin. In further embodiments of the present invention, the shield is moisture resistant and provides for protection from the surrounding environment. This will prevent contaminants from entering the venepunture site causing infection.

In another embodiment of the present invention, the backing of the shield is coated with an adhesive backing that is scored to make at least two removable pieces. In a further embodiment of the present invention, the adhesive backing is scored into two equal halves, with the score line running down a center line of the backing of the shield. The adhesive backing surrounds the body of the intravenous compartment hub and is fixably attached to the skin. In further embodiments of the present invention, the shield is moisture resistant and provides for protection from the surrounding environment. This will prevent contaminants from entering the venepunture site causing infection.

It is another objective of the invention to have a visually safe intravenous shield. Other IV covering are transparent but they are not tamper proof. They need to be covered by a thick wrap (coban) that is hiding the catheter from the patient. Other IV shields are not rip proof and can give the patient free access to the IV site. These covering methods are used regularly and have lead to unsafe IV sites. For example, swelling from Infiltration of intravenous fluids or signs of infection would not be able to be visualized with such a wrap. In yet another embodiment of the present invention, the shield provides for visual safety of the intravenous compartment hub. In a preferred embodiment, the shield is a transparent shield that is tear proof, tamper proof and not easily removed.

In another embodiment of the current invention, the shield is manufactured in an economical and inexpensive manner. Production of the shield provides for a one-piece device.

It is important to note that the present invention is not intended to be limited to a system or method which must satisfy one or more of any stated objects or features of the invention. It is also important to note that the present invention is not limited to the preferred, exemplary, or primary embodiment(s) described herein. Modifications and substitutions by one of ordinary skill in the art are considered to be within the scope of the present invention.

### BRIEF DESCRIPTION OF THE DRAWINGS

These and other features and advantages of the present invention will be better understood by reading the following detailed description, taken together with the drawings wherein

FIG. 1 is a detailed front view of an exemplary embodiment of the present invention;

FIG. 2A is an above view perspective of an exemplary embodiment of the present invention; FIG. 2B is a view from a first side of the protection device; and FIG. 2C is a view from a fourth side of the protection device; and

FIG. 3 is a detailed back view of an exemplary embodiment of the present invention.

### DETAILED DESCRIPTION OF THE PREFERRED EMBODIMENTS

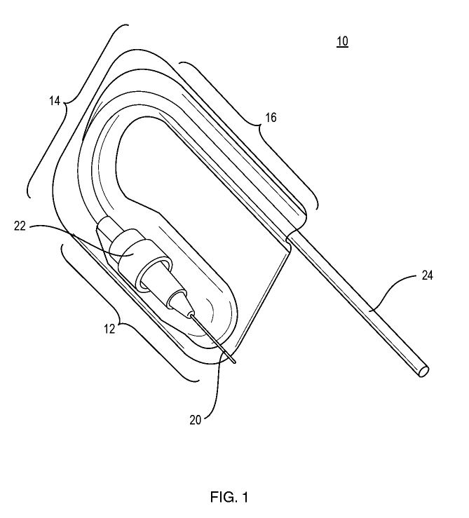

The present invention features an anchoring intravenous shield 10, FIG. 1, which is attachable to the location of an intravenous catheter. An intravenous catheter may be located on the back of the hand, or alternatively at the antecubital site, scalp area, or another location. The anchoring intravenous shield 10 is comprised of three regions, the intravenous compartment hub portion 12, coupled to a U-shaped portion 14, which in turn is coupled to a straight tubing portion 16. The intravenous compartment hub portion 12 is designed to at least partially surround the catheter needle 20 and the luer lock 22. The U-shaped portion 14 and the straight tubing portion 16 are designed to at least partially surround the tube 24 of the catheter. The intravenous compartment hub portion 12 and the straight tubing portion 16 are essentially parallel to one another and are connected by the U-shaped portion 14, which completes essentially a 180 degree turn.

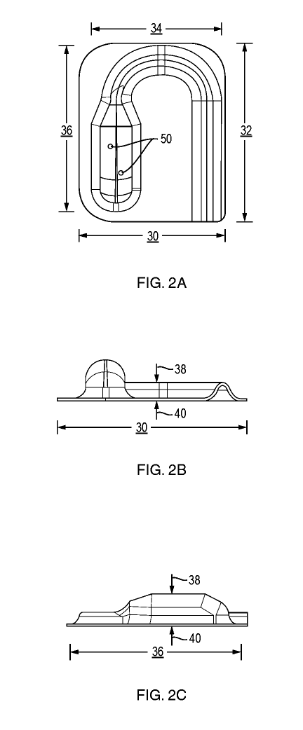

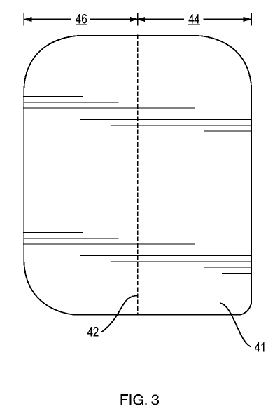

The anchoring intravenous shield 10, FIG. 2A, features four edges, a first edge 30, a second edge 32, a third edge 34, and a fourth edge 36. The anchoring intravenous shield 10 also features two sides, a front side 38, FIG. 2B, and a back side 40. The back side 40 comes into contact with a surface (not shown) of the patient's skin and features an adhesive backing 41, FIG. 3. The adhesive backing 41 typically includes a releasable paper or plastic covering which is preferably scored as shown for example at 42, and which divides and exposes the adhesive backing covering into two pieces, a first adhesive backing piece 44 and a second adhesive backing piece. In a preferred embodiment of the present invention, the adhesive backing is scored along a center line 42 but this is not a limitation of the invention.

The following steps are taken in order to secure the anchoring intravenous shield 10. After insertion of the catheter needle into the patient, a first adhesive backing covering piece is removed and the catheter needle 20 and luer lock 22 are secured in place in the intravenous compartment hub portion 12. A portion of the tube 24 may also be secured at this point. This creates an anchoring quality, which prevents the intravenous catheter from sliding out of the venous lumen and dislodging. Then, the second adhesive covering piece is removed and the intravenous tubing is placed into the U-shaped portion 14 and the tubing portion 16. The adhesive backing 41 is thus now fully secured to the skin of the patient. The anchoring intravenous shield 10 is essentially immovable and maintains the intravenous catheter needle in place. Other methods of adhering the anchoring intravenous shield are within the scope of the present invention.

The anchoring intravenous shield 10 is preferably made from urethane, although other materials are within the scope of the current invention. The urethane is thin, flexible and transparent. The transparency of the material allows for complete visualization of the insertion point, the catheter needle and the luer lock, thereby allowing a medical professional to view the status of the intravenous catheter. The urethane material also allows the anchoring intravenous shield to conform to the shape of the application area, whether it is the hand, or another area of the patient. The urethane material also allows the entire front side to be formed from one-piece of material, which allows for simple manufacture and simple application to a patient. In a preferred embodiment of the present intention, the material is tamper proof and can not be torn. Additionally, the material is preferably moisture proof, contaminant proof, and bacterial proof, thereby keeping the intravenous site safe from harmful substances.

In another embodiment of the present invention, the front side contains one or more breathing holes or apertures 50 to allow moisture and normal skin perspiration to vent outside of the enclosure created by the intravenous compartment hub portion. The breathing holes may be located along the front side of the intravenous compartment hub portion. The front side creates an essentially airtight shield within the closed cover and the breathing holes can be used to allow air circulation. The breathing holes may be circular in shape.

Modifications and substitutions by one of ordinary skill in the art are considered to be within the scope of the present invention.

The invention claimed is:

1. A protective shield for covering an intravenous catheter comprising:
a front side that comprises:
an intravenous compartment hub portion shaped to accommodate at least a portion of a catheter needle and a luer lock;
a U-shaped portion, coupled to said intravenous compartment hub portion, said U-shaped portion shaped to accept a first portion of a tube that is coupled to the luer lock; and
a straight tubing portion, coupled to said U-shaped portion, and shaped to accept a second portion of the tube; and
a generally planar back side, wherein the back side is coated with an adhesive.

2. The protective shield of claim 1 wherein the adhesive coating is covered by a removable protective layer.

3. The protective shield of claim 2 wherein the removable protective layer is scored to make at least two removable pieces.

4. The protective shield of claim 1, wherein the front side of the protective shield is made from a urethane material that is resistant to ripping and tearing.

5. The protective shield of claim 1, wherein the front side of the protective shield is made from a material that is moisture resistant.

6. The protective shield of claim 1, wherein the front side of the protective shield is transparent.

7. The protective shield of claim 1, wherein the front side further includes one or more apertures.
